(19) United States
(12) Patent Application Publication
(10) Pub. No.: US 2022/0211766 A1
(43) Pub. Date: Jul. 7, 2022

(54) SELECTION OF FIBROBLAST DONORS FOR OPTIMIZATION OF ALLOGENEIC FIBROBLAST-MEDIATED REGENERATION

(71) Applicant: Figene, LLC, Houston, TX (US)

(72) Inventors: Pete O'Heeron, Houston, TX (US); Thomas Ichim, San Diego, CA (US)

(21) Appl. No.: 17/594,729

(22) PCT Filed: Apr. 27, 2020
(86) PCT No.: PCT/US2020/030041

§ 371 Date: Oct. 27, 2021

Related U.S. Application Data
(60) Provisional application No. 62/839,644, filed on Apr. 27, 2019.

(51) Int. Cl. A61K 35/33; C12Q 1/6881; G01N 33/569

(52) U.S. Cl. CPC A61K 35/33; C12Q 1/6881; C12Q 2600/158; G01N 2333/70539; G01N 2333/4753; G01N 33/56977

(57) ABSTRACT

Embodiments of the disclosure include methods and compositions related to selecting donor fibroblasts suitable for use in an allogeneic recipient individual. The methods and compositions encompass identifying expression of one or more human leukocyte antigens (HLA) and in some cases expression of one or more other factors, including regenerative factors.

This application claims priority to U.S. Provisional Patent Application Ser. No. 62/839,644, filed Apr. 27, 2019, which is incorporated by reference herein in its entirety.

### TECHNICAL FIELD

Embodiments of the disclosure encompass at least the fields of molecular biology, cell biology, cell therapy, recombinant technology, and medicine.

### BACKGROUND

Fibroblasts comprise the main cell type of connective tissue, possessing a spindle-shaped morphology, whose classical function has historically been believed to produce extracellular matrix responsible for maintaining structural integrity of tissue. Fibroblasts also play an important role in proliferative phase of wound healing, resulting in deposition of extracellular matrix [1, 2]. During wound healing, scar tissue is formed by over-proliferation of fibroblasts. In embryos, and in some types of amphibians, scar-less healing occurs after injury by processes that are currently under intense investigation [3, 4]. With aging, many kinds of tissues and organs undergo fibrosis gradually, such as fibrosis of skin, lung, liver, kidney and heart. The process of scar tissue formation is caused by hyper-proliferation of fibroblasts, as well as these cells producing abnormally large amounts of extracellular matrix and collagens during proliferation and thereby replacing normal organ structure (parenchyma), leading to functional impairment and scar formation, which may further trigger persistent fibrosis.

Fibroblasts were originally considered to possess similar characteristics regardless of their source of origin, a notion that is no longer believed to be entirely accurate [5]. For example, studies have shown that protein antigens such as MHC II [6], C1q receptor [7], LR8 [8], and Thy-1 [9], differ in expression based on tissue origin of fibroblasts. Interestingly, not only origin of fibroblasts affects markers but also proliferating state. For example, one study showed that CD40 expression on fibroblasts was elevated on proliferating fibroblasts but reduced on non-proliferating cells [10]. Other variations in fibroblasts have been detected in various tissues for example, lung fibroblasts are known to possess variable expression of both cell surface marker expression, as well as in their levels of collagen production [11]. Fibroblasts derived from periodontal tissue possess differences in extracellular matrix production, glycogen pools, and morphology [12].

The use of fibroblasts has been primarily restricted to autologous sources, but when used in an allogeneic manner fibroblasts are not matched. The current disclosure provides means of optimization of matching procedures for allogeneic embodiments, as well as selecting donors with enhanced therapeutic activity. The present disclosure provides an advancement in the art of utilizing fibroblasts from allogeneic sources.

### BRIEF SUMMARY

The present disclosure provides methods and compositions for utilization of fibroblasts, including from allogeneic sources when necessary. Specific embodiments provide optimization of fibroblast-matching procedures, including selecting donors, such as with enhanced therapeutic activity.

In particular embodiments, the disclosure encompasses means of matching donors of fibroblast cells with recipients of the fibroblast cells in a manner to increase therapeutic efficacy of allogeneic fibroblast cells. Fibroblast cells may be utilized for a variety of therapeutic indications including at least immune modulatory, angiogenic, neurogenic, anti-apoptotic, chondrogenic, and/or hepatogenic applications, as examples.

In specific embodiments, the disclosure pertains to the field of cell transplantation, more specifically, the disclosure pertains to means of generating cells useful for transplantation such as for therapeutic indications, more specifically, the disclosure pertains to means of matching donors and recipients, and additionally the disclosure pertains to selecting donors possessing an increased therapeutic index.

In one embodiment, there is a method of selecting donor fibroblast cells and/or derivatives and/or vesicles thereof to provide to one or more recipient individuals, comprising the steps of: identifying the expression of one or more human leukocyte antigens (HLA) on said donor fibroblast cells and/or derivatives and/or vesicles thereof; and matching one or more recipient individuals to the donor fibroblast cells and/or derivatives and/or vesicles thereof based on the expression of one or more HLA in the recipient. The expression of the HLA may be determined by nucleic acid and/or protein levels. In some cases, the HLA is HLA-A, HLA-B, HLA-C, HLA-C, HLA-DP, HLA-DQ, HLA-DR, HLA-B27, or a combination thereof.

In any method encompassed herein, donor fibroblast cells and/or derivatives and/or vesicles thereof are further analyzed for one or more additional functional properties and/or one or more additional genotypes. For example, the donor fibroblast cells and/or derivatives and/or vesicles thereof may be analyzed for having one or more regenerative properties, including expressing one or more regenerative factors. Examples of the one or more regenerative factors may be selected from the group consisting of interleukin (IL)-1, IL-3, granulocyte colony-stimulating factor (G-CSF), granulocyte macrophage colony-stimulating factor (GM-CSF), macrophage colony-stimulating factor (M-CSF), thrombopoietin (TPO), leukemia inhibitory factor, hepatic growth factor (HGF), brain derived neurotrophic factor (BDNF), nerve growth factor (NGF), connective tissue growth factor (CTGF), vascular endothelial growth factor (VEGF), fibroblast growth factor a (FGFa), fibroblast growth factor b (FGFb), platelet derived growth factor AA (PDGF-AA), platelet derived growth factor AB (PDGF-AB), angiopoietin, and a combination thereof. The donor fibroblast cells and/or derivatives and/or vesicles thereof may be selected for a particular therapeutic application based on the expression of one or more regenerative or other factors. In some cases, the therapeutic application is to stimulate hematopoiesis, and in specific cases the one or more regenerative or other factors are selected from the group consisting of interleukin-1 (IL-1), IL-3, granulocyte-colony stimulating factor (G-CSF), granulocyte-macrophage colony-stimulating factor (GM-CSF), thrombopoietin (TPO), leukemia inhibitory factor, and a combination thereof. In certain cases, the therapeutic application is to stimulate neurogenesis, and in specific cases, the one or more regenerative or other factors are selected from the group consisting of brain derived neurotrophic factor (BDNF), nerve growth factor (NGF), connective tissue growth factor (CTCF), and a combination thereof. The therapeutic application may be to stimulate angiogenesis, and in some cases the one or more regenerative or other factors may be selected from the group consisting of vascular endothelial growth factor (VEGF), fibroblast growth factor A (FGF-A), fibroblast growth factor B (FGF-B), platelet-derived growth factor AA (PDGF-AA), platelet-derived growth factor AB (platelet-derived growth factor AB), angiopoietin, and a combination thereof. In cases wherein the therapeutic application is to stimulate hepatic regeneration, the one or more regenerative or other factors is hepatic regeneration factor (HGF).

Donors related to the methods of the disclosure may be a mammal, such as a human, primate, murine, canine, feline, porcine, and/or bovine donor.

Embodiments of the disclosure include compositions of fibroblast cells and/or derivatives and/or vesicles thereof selected from any method encompassed by the disclosure. Pharmaceutical compositions may comprise the compositions and include a pharmaceutically acceptable carrier.

In certain embodiments, there is a method of treating a medical condition in an individual comprising the step of delivering to the individual a therapeutically effective amount of one or more pharmaceutical compositions encompassed by the disclosure. The individual may have or is at risk of having an inflammatory condition and/or a neurodegenerative condition and/or an autoimmune condition and/or a neoplastic condition and/or the frailty of aging, as examples.

It is specifically contemplated that any limitation discussed with respect to one embodiment of the disclosure may apply to any other embodiment of the disclosure. Furthermore, any composition of the disclosure may be used in any method of the disclosure, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Summary of Invention, Detailed Description of the Embodiments, and Claims.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### DETAILED DESCRIPTION

### I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the disclosure may “consist essentially of” or “consist of” one or more sequences of the invention, for example. Some embodiments may consist of or consist essentially of one or more elements, method steps, and/or methods of the invention. It is contemplated that any method or composition described herein can be implemented with respect to any other method or composition described herein. The scope of the present application is not intended to be limited to the particular embodiments of the process, machine, manufacture, composition of matter, means, methods and steps described in the specification.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe etc. A second exemplary method of administering is by a direct mechanism such as, local tissue administration, oral ingestion, transdermal patch, topical, inhalation, suppository etc.

As used herein “affecting the expression” and “modulating the expression” of a protein or gene, as used herein, should be understood as regulating, controlling, blocking, inhibiting, stimulating, enhancing, activating, mimicking, bypassing, correcting, removing, and/or substituting said expression, in more general terms, intervening in said expression, for instance by affecting the expression of a gene encoding that protein.

As used herein “allogeneic” refers to tissues or cells from another body that in a natural setting are immunologically incompatible or capable of being immunologically incompatible, although from one or more individuals of the same species.

As used herein, the terms “allostimulatory” and “alloreactive” refer to stimulation and reaction of the immune system in response to an allologous antigens, or “alloantigens” or cells expressing a dissimilar HLA haplotype.

As used herein, the term “autoimmunity” refers to the system of immune responses of an organism against its own healthy cells and tissues.

As used herein, “autologous” refers to tissues or cells that are derived or transferred from the same individual's body (i.e., autologous blood donation; an autologous bone marrow transplant).

As used herein, the term “autotransplantation” refers to the transplantation of organs, tissues, and/or cells from one part of the body in an individual to another part in the same individual, i.e., the donor and recipient are the same individual. Tissue transplanted by such “autologous” procedures is referred to as an autograft or autotransplant.

As used herein “cell culture” or “culture” or “cultured” refers to an artificial in vitro system containing viable cells, whether quiescent, senescent or (actively) dividing. In a cell culture, cells are grown and maintained at an appropriate temperature, typically a temperature of 37° C. and under an atmosphere typically containing oxygen and CO₂. Culture conditions may vary widely for each cell type though, and variation of conditions for a particular cell type can result in different phenotypes being expressed.

As used herein, the terms “antibody” and “antibodies” refer to monoclonal antibodies, multispecific antibodies, synthetic antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to a polypeptide antigen encoded by a gene comprised in the genomic regions or affected by genetic. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The term “delivering” or “delivered as used herein, refers to any method of providing a composition(s) to an individual such that the composition has its intended effect on the patient. For example, one method of administering is by an indirect mechanism using a medical device such as, but not limited to a catheter, applicator gun, syringe, etc. A second exemplary method of administering is by a direct mechanism such as, local tissue administration, oral ingestion, transdermal patch, topical, inhalation, suppository, etc.

The term “derivative” as used herein refers to exosomes, microvesicles, apoptotic bodies, conditioned media, and so forth that come from fibroblasts. In specific cases, it refers to materials that are secreted from fibroblasts during growth and culturing of the cells. For conditioned media, fibroblasts may be cultured in a suitable growth media in order to obtain conditioned media. Fibroblast cells for obtaining conditioned media can undergo at least 25, 30, 35, or 40 doublings, for example prior to reaching a senescent state. Methods for deriving cells capable of doubling to reach 10¹⁴ cells or more are encompassed herein. Certain methods may be used that derive cells that can double sufficiently to produce at least about 1014, 1015, 1016, or 10¹⁷ or more cells when seeded at from about 10³ to about 10⁶ cells/cm² in culture, as one example. Particularly, these cell numbers are produced within 80, 70, or 60 days or less. In one embodiment, fibroblast cells used for the generation of conditioned media are isolated and expanded.

As used herein “differentially present” refers to differences in the quantity or frequency (incidence of occurrence) of at least one marker present in a sample taken from a test subject as compared to a control subject (or a recipient subject as compared to a donor subject). For example, a marker can be a gene expression product that is present at an elevated level or at a decreased level in blood samples of one or more risk subjects compared to samples from one or more control subjects. Alternatively, a marker can be a gene expression product that is detected at a higher frequency or at a lower frequency in samples of blood from one or more risk subjects compared to samples from one or more control subjects. In some embodiments, a gene expression product is “differentially present” between two samples if the amount of the gene expression product in one sample is statistically significantly different from the amount of the gene expression product in the other sample. For example, a gene expression product is differentially present between two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

The term “fibrosis” means the formation of excessive fibrous connective tissue in an organ or tissue. Fibrosis occurs in normal physiology to act as a deposit of connective tissue. In pathology, fibrosis can be used to describe an excess state of deposition of extracellular material and proteins that can result in scarring, thickening of the afflicted tissue, and interfere with the normal function of the tissue or organ.

As used herein “Immunoassay” is an assay that uses an antibody to specifically bind an antigen (e.g., a marker). The immunoassay is characterized by the use of specific binding properties of a particular antibody to isolate, target, and/or quantify the antigen. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The term “individual” and “subject” that may be used interchangeably, as used herein, refer to a human or animal that may or may not be housed in a medical facility and may be treated as an outpatient of a medical facility. The individual may or may not be receiving one or more medical compositions from a medical practitioner and/or via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method and/or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

As used herein “matching” refers to the degree of similarity between the genetic makeup of a cell product or unit to be used for therapeutic and/or prophylactic purpose (including vaccination or inducing an immune response) into an individual and the individual's genetic makeup. For the purposes of this disclosure, when two individuals share a type, they are said to be a match, meaning that their tissues are immunologically compatible with each other. The degree to which blood parameters need be identical will vary from patient to patient, and from year to year depending on the current state of technology. Matching then refers to providing the desired degree of match. For example, bone marrow and peripheral blood stem cell transplantation requires a greater degree of matching than blood cord stem cell transplantation. Matching can refer to a match with about 90%, 80%, 70%, 60%, 50%, or 40% similarity based on HLA matching; HLA matching refers to the number of HLA alleles that are similar between the donor and the recipient. A matching fibroblast unit is one that is from a donor not related to the potential recipient.

The term “pharmaceutically” or “pharmacologically acceptable,” as used herein, refer to molecular entities and compositions that do not produce adverse, allergic, or other untoward reactions when administered to an animal or a human.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject. In specific embodiments, the onset of one or more symptoms is delayed.

As used herein “specifically (or selectively) binds” when referring to an antibody, or “specifically (or selectively) immunoreactive with”, when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein.

“Therapeutic agent” means to have “therapeutic efficacy” in modulating angiogenesis and/or wound healing and an amount of the therapeutic is said to be a “angiogenic modulatory amount”, if administration of that amount of the therapeutic is sufficient to cause a significant modulation (i.e., increase or decrease) in angiogenic activity when administered to a subject (e.g., an animal model or human patient) needing modulation of angiogenesis.

As used herein, the term “therapeutically effective amount” is synonymous with “effective amount,” “therapeutically effective dose,” and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells.

As used herein, the term “transplantation” refers to the process of taking living tissue or cells and implanting it in another part of the body or into another body.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

As used herein “type or “typing” as used herein refers to any and all characteristics of a sample, e.g., endothelial or endothelial progenitor cell product sample, which might be of relevance or importance for any potential use of the sample. The term and the corresponding testing conducted to determine the “type” of the sample is thus not limited to any particular tests mentioned herein, e.g., HLA typing. Determination of which tests are relevant and how to perform them may be entirely conventional and may change with technological developments. Thus the term “type identifier” refers to any characteristic that can be used for identification purposes.

### II. General Embodiments

Disclosed are methods and compositions for identifying fibroblast cells and/or donors thereof as sources of allogeneic cells, such as for cell therapy. Embodiments include methods of selecting donors for derivation of fibroblasts to be used for any purpose. Thus, in specific embodiments fibroblasts are obtained from one or more individuals that are suited for one or more particular purposes because of one or more characteristics that they comprise. Those fibroblasts, and/or cells and/or vesicles derived from those fibroblasts, are utilized for a cell therapy and/or for producing a therapy (such as one or more components obtained from the derived fibroblasts and/or derivative and/or vesicles, for example).

In specific cases, the fibroblasts are to be utilized for regenerative purposes in tissue and/or organs of an individual in need thereof. In one embodiment, fibroblast donor(s) are selected based on human leukocyte antigen (HLA) matching between donor(s) and recipient(s). In an additional or alternative embodiment, donor(s) are selected based on the ability of fibroblasts to exert one or more certain desired therapeutic properties. In a particular embodiment, fibroblasts are selected based on expression of one or more molecules associated with one or more certain therapeutic characteristics.

Embodiments of the disclosure encompass method of selecting donor fibroblast cells and/or derivatives and/or vesicles thereof to provide to one or more recipient individuals, comprising the steps of identifying the expression of one or more human leukocyte antigens (HLA) on the donor fibroblast cells and/or derivatives and/or vesicles thereof; and identifying one or more recipient individuals for the donor fibroblast cells and/or derivatives and/or vesicles thereof based on the expression of one or more HLA in the recipient. The donor is selected based upon expression of matching the expression of one or more HLA in the recipient.

In one embodiment of the disclosure, donor(s) are selected based on ability of fibroblasts from the donor(s) to produce one or more regenerative factors. The disclosure encompasses assessment of one or more regenerative factors (including the level of one or more factors) as a means of quantifying fibroblast potency. The expression level of regeneration-related factor(s) may be determined in cell culture media and/or cell extracts, as one example. In a certain case, the testing provides quantitative or semi-quantitative results, which facilitate comparison with reference levels of expression and/or a model profile, for example one correlated to a treatment regimen and subject outcome.

In particular embodiments, methods utilize selection of donors for the ability of their cells to produce high levels of one or more therapeutic growth factors or one or more regenerative factors. Regenerative factors useful for quantification depend on the type of regenerative effect that is desired. In embodiments of the disclosure in which regeneration refers to stimulation of hematopoiesis, the regenerative factors include at least interleukin (IL)-1, IL-3, granulocyte colony-stimulating factor (G-CSF), granulocyte macrophage colony-stimulating factor (GM-CSF), macrophage colony-stimulating factor (M-CSF), thrombopoietin (TPO), leukemia inhibitory factor, or a combination thereof. In situations where hepatic regeneration is desired, assessment of factors such as hepatic growth factor (HGF) may be assessed. In situations where neurogenesis is desired, assessment of factors such as brain derived neurotrophic factor (BDNF), nerve growth factor (NGF), and connective tissue growth factor (CTGF) is assessed. In situations where angiogenesis is desired, factors such as vascular endothelial growth factor (VEGF), fibroblast growth factor a (FGFa), fibroblast growth factor b (FGFb), platelet derived growth factor AA (PDGF-AA), platelet derived growth factor AB (PDGF-AB), and/or angiopoietin may be assessed.

The testing of factor(s) may comprise measuring the presence and/or expression levels of the factor, either within the cell or secreted into the cell culture medium, using any suitable assay, such as an antibody-based assay, for example Western blotting or immunocytochemistry, but preferably using quantitative immunoassays such as ELISA. Kits for measuring levels of many proteins using ELISA methods are commercially available (e.g. from R&D Systems) and ELISA methods can be developed using well known techniques. Antibodies for use in such ELISA methods either are commercially available or may be prepared using well known methods. The testing may comprise measurement of the levels of gene expression at the mRNA level, using quantitative mRNA amplification methods such as RT-PCR, isothermal nucleic acid amplification, or variants thereof. Systems for carrying out these methods also are commercially available, for example the TaqMan® system (Roche Molecular System, Alameda, Calif.) and the Light Cycler system (Roche Diagnostics, Indianapolis, Ind.). Methods for devising appropriate primers for use in RT-PCR and related methods are well known in the art. Angiogenesis-related factor or other factor protein expression levels may be correlated with mRNA levels by quantitative RT-PCR. Nucleic acid arrays may be used to study the expression of one or more angiogenesis-related factors. In particular, arrays provide a method for simultaneously assaying expression of a large number of genes. Such methods are now well known in the art and commercial systems are available from, for example, Affymetrix (Santa Clara, Calif.), Incyte (Palo Alto, Calif.), Research Genetics (Huntsville, Ala.) and Agilent (Palo Alto, Calif.). The disclosure further provides an array of polynucleotide probes, the array comprising a support with at least one surface and a plurality of different polynucleotide probes, wherein each different polynucleotide probe hybridizes under stringent hybridization conditions to a gene product.

Other methods of quantitative analysis of protein expression levels may be used which include proteomics technologies such as isotope coded affinity tag reagents, MALDI TOF/TOF tandem mass spectrometry and 2D-gel/mass spectrometry technologies. In one particular embodiment cell extracts may be used to probe a proteome array (e.g. Proteome Profiler Array obtained from R&D Systems) that contains capture antibodies specific to one or more regeneration-related factors.

Prior to testing, the cells may be isolated from tissue using conventional separation and differentiation techniques. In one embodiment, the cells are expanded in cell culture. The methods of the disclosure may comprise culturing the cells from the sample obtained from the each subject. The cells from the cell culture may be exposed to one or more agonists or one or more antagonists of angiogenesis. The cells may be maintained under normoxic or hypoxic culture conditions. The cells may be maintained in conditions of hypoxic and/or normoxic culture conditions and exposed to one or more agonists or one or more antagonists of regenerative activity in sequence or in parallel, and the expression levels of the regeneration-related factor(s) may be tested in both. A hypoxic condition or environment may be about 0.5% to about 15% oxygen, such as from about 1% to about 5% oxygen. Normoxic conditions include conditions at about 18% to about 23% oxygen, such as about 21%. In particular embodiments, the cells obtained from the subject are expanded in culture and tested for expression levels of one or more regenerative factors.

In a specific embodiment, ascertaining the expression of TWIST is utilized to select a certain type of desired fibroblasts from donors. In a specific embodiment, fibroblasts from donors that naturally express high Twist are utilized if immune modulation activity is desired. In one embodiment, immune modulation activity is the ability to produce interleukin-10 and/or the ability to induce generation of T regulatory cells. Elevated expression may be the level compared to a pool of age matched controls. For example, out of 10 donors, 2 may possess at least more than 25% Twist protein expression as compared to the average of the 10 donors. In some embodiments, if a higher differentiation ability of fibroblasts is desired, donors are selected with low Twist expression. Low expression of Twist may be the level compared to a pool of age matched controls. For example, out of 10 donors, 2 may possess at least less than 25% Twist protein expression as compared to the average of the 10 donors. In a specific case, valproic acid suppresses Twist and makes fibroblast cells more amenable to differentiation. One example of Twist polynucleotide is in the GenBank® Accession No. at NM_000474 and one example of Twist polypeptide is in the GenBank® Accession No. NP_000465. The expression of Twist may be ascertained at any level, including mRNA and/or protein, for example.

In one embodiment of the disclosure, matching is performed to obtain appropriate cells to treat one or more inflammatory conditions in a recipient. In such cases, the term “inflammatory conditions” is an inclusive term and includes, for example: (1) tissue damage from ischemia-reperfusion following acute myocardial infarction, aneurysm, stroke, hemorrhagic shock, crush injury, multiple organ failure, hypovolemic shock intestinal ischemia, spinal cord injury, and traumatic brain injury; (2) inflammatory disorders, e.g., burns, endotoxemia and septic shock, adult respiratory distress syndrome, cardiopulmonary bypass, hemodialysis; anaphylactic shock, severe asthma, angioedema, Crohn's disease, sickle cell anemia, poststreptococcal glomerulonephritis, membranous nephritis, and pancreatitis; (3) transplant rejection, e.g., hyperacute xenograft rejection; (4) pregnancy related diseases such as recurrent fetal loss and pre-eclampsia, and (5) adverse drug reactions, e.g., drug allergy, IL-2 induced vascular leakage syndrome and radiographic contrast media allergy. Complement-mediated inflammation associated with autoimmune disorders including, but not limited to, myasthenia gravis, Alzheimer's disease, multiple sclerosis, rheumatoid arthritis, systemic lupus erythematosus, insulin-dependent diabetes mellitus, acute disseminated encephalomyelitis, Addison's disease, antiphospholipid antibody syndrome, autoimmune hepatitis, Crohn's disease, Goodpasture's syndrome, Graves' disease, Guillain-Barre syndrome, Hashimoto's disease, idiopathic thrombocytopenic purpura, pemphigus, Sjogren's syndrome, and Takayasu's arteritis, may also be treated with cells identified by the methods described herein.

In some embodiments of the disclosure, matching of donor fibroblasts, and/or selection of fibroblasts is performed in order to treat a neurodegenerative condition. The “neurodegenerative condition” (or disorder) is an inclusive term encompassing acute and chronic conditions, disorders or diseases of the central or peripheral nervous system. A neurodegenerative condition may be age-related, or it may result from injury or trauma, or it may be related to a specific disease or disorder. Acute neurodegenerative conditions include, but are not limited to, conditions associated with neuronal cell death or compromise including cerebrovascular insufficiency, e.g. due to stroke, focal or diffuse brain trauma, diffuse brain damage, spinal cord injury or peripheral nerve trauma, e.g., resulting from physical or chemical burns, deep cuts or limb severance. Examples of acute neurodegenerative disorders are: cerebral ischemia or infarction including embolic occlusion and thrombotic occlusion, reperfusion following acute ischemia, perinatal hypoxic-ischemic injury, cardiac arrest, as well as intracranial hemorrhage of any type (such as epidural, subdural, subarachnoid and intracerebral), and intracranial and intravertebral lesions (such as contusion, penetration, shear, compression and laceration), as well as whiplash and shaken infant syndrome. Chronic neurodegenerative conditions include, but are not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (including, but not limited to Creutzfeldt-Jakob, Gerstmann-Straussler-Scheinker disease, Kuru and fatal familial insomnia), demyelination diseases and disorders including multiple sclerosis and hereditary diseases such as leukodystrophies.

The fibroblasts for use in the current disclosure are of any mammalian origin e.g. human, rat, primate, porcine and the like. In one embodiment of the disclosure, the fibroblasts are derived from human umbilicus. umbilicus-derived cells, cells are capable of self-renewal and expansion in culture, and have the potential to differentiate into cells of other phenotypes.

As one example, methods of deriving cord tissue fibroblast cells from human umbilical tissue are provided. The cells are capable of self-renewal and expansion in culture and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) re-suspending the tissue in a culture medium, and (e) providing growth conditions that allow for the growth of a human umbilicus-derived cell capable of self-renewal and expansion in culture and having the potential to differentiate into cells of other phenotypes. Tissue can be obtained from any completed pregnancy, term or less than term, whether delivered vaginally, or through other routes, for example from surgical Cesarean section. Obtaining tissue of any kind from tissue banks is also considered within the scope of the present disclosure.

The tissue may be rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In one embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently considered are enzyme activites selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also considered are methods that include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other embodiments, the tissue is incubated at 37° C. during the enzyme treatment of the dissociation step. Diluting the digest may also improve yields of cells as cells may be trapped within a viscous digest.

While the use of enzyme activities is presently considered, it is not required for isolation methods as provided herein. Methods based on mechanical separation alone may be successful in isolating the instant cells from the umbilicus as discussed above. The cells can be re-suspended after the tissue is dissociated into any culture medium as discussed herein above. Cells may be re-suspended following a centrifugation step to separate out the cells from tissue or other debris. Resuspension may involve mechanical methods of re-suspending, or simply the addition of culture medium to the cells.

Providing the growth conditions allows for a wide range of options as to culture medium, supplements, atmospheric conditions, and relative humidity for the cells. A particular temperature is 37° C., however the temperature may range from about 35° C. to 39° C. depending on the other culture conditions and desired use of the cells or culture.

Presently considered are methods that provide cells that require no exogenous growth factors, except as are available in the supplemental serum provided with the Growth Medium. Also provided herein are methods of deriving cells capable of expansion in the absence of particular growth factors. The methods may require that the particular growth factors (for which the cells have no requirement) be absent in the culture medium in which the cells are ultimately resuspended and grown in. In this sense, the method is selective for those cells capable of division in the absence of the particular growth factors. Particular cells in some embodiments are capable of growth and expansion in chemically-defined growth media with no serum added. In such cases, the cells may require certain growth factors, which can be added to the medium to support and sustain the cells. Presently considered factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10¹⁴ cells or more are provided. Preferred are those methods which derive cells that can double sufficiently to produce at least about 10¹⁴, 10¹⁵, 10¹⁶, or 10¹⁷ or more cells when seeded at from about 10³ to about 10⁶ cells/cm² in culture. Preferably these cell numbers are produced within 80, 70, or 60 days or less. In one embodiment, cord tissue fibroblast cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR, DP, DQ.

In some embodiments, fibroblasts are collected from donors and information about each donation is recorded. In some specific embodiments, the recorded information comprises at least some data selected from the group consisting of the type of cells, their tissue of origin, the date of their collection and the identity of the donor. In other specific embodiments, the recorded information comprises results obtained from various characterization assays. Examples include HLA typing, determining the presence of specific markers, determining specific SNP alleles and/or performing a nucleated cell count on the stem cell unit. In some embodiments, the collected cells are sorted according to at least one criterion. In some specific embodiments, they are sorted according to their type, their tissue of origin, the date of their collection and the donor identity.

In some embodiments, the collected fibroblasts are stored under appropriate conditions to keep the fibroblast cells viable and functional, although in other embodiments they are used without having to store the cells. In some specific embodiments, the fibroblasts are stored under cryopreservation conditions. In other embodiments, the fibroblasts are stored in the bank are for allogeneic use. In some embodiments, the stored fibroblast cells are used for allogeneic transplantations. In other embodiments, the stored fibroblasts are used for the establishment of cell lines having, for example, good viability and other desirable characteristics for research and pharmaceutical applications.

In some embodiments, the fibroblasts are stored in the bank are arranged in units. According to these embodiments, each donation to the bank (each deposit of fibroblasts) is divided into a plurality of units. In some typical embodiments, a unit comprises a population of fibroblasts of the same type that were collected from a single donor in a single donation. In some exemplary embodiments, a unit includes fibroblasts expressing a specific marker or markers. In some embodiments, a unit is further defined by the number of nucleated cells present in the sample. Upon request, one or more units may be allocated to a subject in need thereof. In some embodiment, a fraction of a unit is allocated to a recipient in need. In some typical embodiments, the number of units to be allocated depends on the number of nucleated cells in each unit and the medical condition to be treated. In some embodiments, the amount of fibroblasts, or the number of units, available for allocation to an individual depends on the amount of donations made.

In some embodiments, the fibroblasts can be subjected to further processing after their collection. In some specific embodiments, the collected fibroblasts can be cultured, expanded and/or proliferated. In additional specific embodiments, the collected fibroblasts are processed in order to achieve therapeutic levels. In some embodiments, an optimal combination of fibroblasts can be selected from the reservoir of cells, in order to treat a certain pathological condition. As one example, the fibroblasts may be transfected with one or more exogenous gene products, including on a vector (viral or non-viral), for example. The gene product may be of any kind, including one or more therapeutic proteins and/or one or more gene products that enhances activity of the fibroblasts or renders the fibroblasts therapeutic or to have an enhanced therapeutic activity.

According to another aspect, the present disclosure provides a method of fibroblast banking, the method comprising periodically collecting a plurality of donations from an individual throughout the individual's life. In some embodiments, the method comprises collecting fibroblasts from more than one source. In some embodiments, the method comprises collecting fibroblasts of more than one type, whether or not from the same individual.

In some embodiments of the disclosure, donor cells are modulated to possess enhanced therapeutic properties.

In some embodiments of the disclosure, fibroblasts are transfected to possess enhanced neuromodulatory and neuroprotective properties. The transfection may be accomplished by use of non-viral or viral vectors, including for example lentiviral vectors; the means to perform lentiviral mediated transfection are well-known in the art and discussed in the following references [13-19]. Some specific examples of lentiviral based transfection of genes into fibroblasts include transfection of stromal derived factor 1 (SDF-1) to promote stem cell homing, particularly hematopoietic stem cells [20], glial cell line-derived neurotrophic factor (GDNF) to treat Parkinson's in an animal model [21], hepatic growth factor (HGF) to accelerate re-myelination in a brain injury model [22], protein kinase B (Akt) to protect against pathological cardiac remodeling and cardiomyocyte death [23], tissue necrosis factor (TNF)-related apoptosis-inducing ligand (TRAIL) to induce apoptosis of tumor cells [24-27], PGE-1 synthase for cardioprotection [28], nerve growth factor IB (NUR77) to enhance migration [29], brain-derived neurotrophic factor (BDNF) to reduce ocular nerve damage in response to hypertension [30], hypoxia-induced factor-1 (HIF-1) alpha to stimulate osteogenesis [31], dominant negative chemokine ligand 2 (CCL2) to reduce lung fibrosis [32], interferon beta to reduce tumor progression [33], major histocompatibility complex, class I, G (HLA-G) to enhance immune suppressive activity [34], human telomerase reverse transcriptase (hTERT) to induce differentiation along the hepatocyte lineage [35], cytosine deaminase [36], octamer-binding transcription factor 4 (OCT-4) to reduce senescence [37, 38], bone morphogenetic protein (BMP) and activin membrane-bound inhibitor homolog (BAMBI) to reduce tissue growth factor (TGF) expression and protumor effects [39], HO-1 for radioprotection [40], tumor necrosis factor superfamily member 14 (TNFSF14) (LIGHT) to induce antitumor activity [41], miR-126 to enhance angiogenesis [42, 43], B-cell lymphoma 2 (bcl-2) to induce generation of nucleus pulposus cells [44], telomerase to induce neurogenesis [45], C-X-C chemokine receptor type 4 (CXCR4) to accelerate hematopoietic recovery [46] and reduce unwanted immunity [47], wnt11 to promote regenerative cytokine production [48], and the human growth factor (HGF) antagonist NK4 to reduce cancer [49]. Neuroprotective activity of fibroblasts may be demonstrated by administration of cells into the middle cerebral artery ligation model in which administration of cells intravenously in the rat model results in reduced infarct size and superior recovery to controls. These results are achieved by intravenous administration fibroblasts at 50,000 to 10 million cells per animal, such as 1-4 million cells per animal.

Specific embodiments of the disclosure encompass methods of matching a fibroblast donor with a fibroblast recipient by identifying one or more human leukocyte antigens (HLA) expressed on donor-derived cells and selecting a recipient possessing the same or similar HLA expression. Embodiments of the disclosure include methods of matching a fibroblast donor with a fibroblast recipient, the method comprising the steps of: a) obtaining a population of cells from a fibroblast donor; b) identifying one or more HLA expressed on donor derived cells; and c) selecting a donor possessing closest homology of HLA with said donor. Homology is determined by the number of matching alleles. The matching may be performed using antibody or gene typing of HLA alleles. Rules and guidance for HLA matching are known in the art for hematopoietic transplantation and are described such as at the website for the Department of Health and Human Services, Organ Procurement and Transplantation Network webpage.

In certain cases, the fibroblasts are derived from a source of tissue selected from the group consisting of: a) adipose; b) dermal; c) placental; d) hair follicle; e) keloid; f) bone marrow; g) peripheral blood; h) umbilical cord; i) foreskin; and j) a combination thereof. The fibroblast donors may be derived from a variety of genetic backgrounds, including from a variety of ethnic diversities, genders, ages, and so forth.

The HLA genes may or may not be identified based on DNA sequence and/or RNA sequence and/or protein amino acid sequence. In at least some cases, homology between donor and recipient may be identified based on homology between antigenic determinants, such as HLA alleles including one or more of HLA-A, HLA-B, HLA-C, HLA-DP, HLA-DQ, HLA-DR, HLA-B27. One or more particular HLA alleles may or may not be identified by antibodies. One or more HLA alleles may be identified by genotyping. In specific embodiments, a donor is selected for use in generation of regenerative fibroblasts where the donor is selected from a plurality of donors. In specific embodiments, a donor with the highest regenerative property may be chosen from a plurality of donors. Although the regenerative property may be of any kind, in specific embodiments the regenerative property is for angiogenesis, hepatogenesis, neurogenesis, chondrogenic, and/or hematopoiesis.

### III. Pharmaceutical Compositions

Pharmaceutical compositions of the present disclosure comprise an effective amount of donor fibroblast cells and/or derivatives and/or vesicles (such as exosomes) thereof dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that comprises at least donor fibroblast cells and/or derivatives and/or vesicles thereof will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical composition may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The donor fibroblast cells and/or derivatives and/or vesicles thereof may be formulated into a composition in any form. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include the cells, derivatives, and/or vesicles and one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the cells, derivatives, and/or vesicles may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 50,000 cells to 500 million cells per kilogram, more specifically 100,000-1 million cells per kilogram, and more specifically approximately 500,000-1 million cells per kilogram, such as when administrated intravenously.

A. Alimentary Compositions and Formulations

In particular embodiments of the present disclosure, the cells, derivatives, and/or vesicles are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the cells, derivatives, and/or vesicles may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, cells, derivatives, and/or vesicles may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the cells, derivatives, and/or vesicles may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present disclosure for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### IV. Kits of the Disclosure

Any of the cellular and/or non-cellular compositions described herein or similar thereto may be comprised in a kit. In a non-limiting example, one or more reagents for use in methods for preparing cellular therapy may be comprised in a kit. Such reagents may include cells, one or more growth factors, vector(s) one or more costimulatory factors, media, enzymes, buffers, nucleotides, antibodies of any kind, salts, primers, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

extracting one or more samples from an individual. The apparatus may be a syringe, fine needles, scalpel, swab, scraper, and so forth.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
1. Landen, N. X., D. Li, and M. Stahle, Transition from inflammation to proliferation: a critical step during wound healing. Cell Mol Life Sci, 2016. 73(20): p. 3861-85.
2. Reinke, J. M. and H. Sorg, Wound repair and regeneration. Eur Surg Res, 2012. 49(1): p. 35-43.
3. Ho, S., H. Marcal, and L. J. Foster, Towards scarless wound healing: a comparison of protein expression between human, adult and foetal fibroblasts. Biomed Res Int, 2014. 2014: p. 676493.
4. Adzick, N. S. and M. T. Longaker, Scarless fetal healing. Therapeutic implications. Ann Surg, 1992. 215(1): p. 3-7.
5. Chang, Y., H. Li, and Z. Guo, Mesenchymal stem cell-like properties in fibroblasts. Cell Physiol Biochem, 2014. 34(3): p. 703-14.
6. Phipps, R. P., et al., Characterization of two major populations of lung fibroblasts: distinguishing morphology and discordant display of Thy 1 and class H MHC. Am J Respir Cell Mol Biol, 1989. 1(1): p. 65-74.
7. Akamine, A., G. Raghu, and A. S. Narayanan, Human lung fibroblast subpopulations with different C1q binding and functional properties. Am J Respir Cell Mol Biol, 1992. 6(4): p. 382-9.
8. Etikala, A., et al., LR8 Expression in fibroblasts of healthy and fibrotic human tissues. Biochem Biophys Rep, 2017. 10: p. 165-171.
9. Koumas, L., et al., Fibroblast heterogeneity: existence of functionally distinct Thy 1(+) and Thy 1(−) human female reproductive tract fibroblasts. Am J Pathol, 2001. 159(3): p. 925-35.
10. Fries, K. M., et al., CD40 expression by human fibroblasts. Clin Immunol Immunopathol, 1995. 77(1): p. 42-51.
11. Fries, K. M., et al., Evidence of fibroblast heterogeneity and the role of fibroblast subpopulations in fibrosis. Clin Immunol Immunopathol, 1994. 72(3): p. 283-92.
12. Lekic, P. C., N. Pender, and C. A. McCulloch, Is fibroblast heterogeneity relevant to the health, diseases, and treatments of periodontal tissues? Crit Rev Oral Biol Med, 1997. 8(3): p. 253-68.
13. Zhang, X. Y., et al., Lentiviral vectors for sustained transgene expression in human bone marrow-derived stromal cells. Mol Ther, 2002. 5(5 Pt 1): p. 555-65.
14. Kyriakou, C. A., et al., Human mesenchymal stem cells (hMSCs) expressing truncated soluble vascular endothelial growth factor receptor (tsFlk-1) following lentiviral-mediated gene transfer inhibit growth of Burkitt's lymphoma in a murine model. J Gene Med, 2006. 8(3): p. 253-64.
15. Worsham, D. N., et al., In vivo gene transfer into adult stem cells in unconditioned mice by in situ delivery of a lentiviral vector. Mol Ther, 2006. 14(4): p. 514-24.
16. Rabin, N., et al., A new xenograft model of myeloma bone disease demonstrating the efficacy of human mesenchymal stem cells expressing osteoprotegerin by lentiviral gene transfer. Leukemia, 2007. 21(10): p. 2181-91.
17. Kallifatidis, G., et al., Improved lentiviral transduction of human mesenchymal stem cells for therapeutic intervention in pancreatic cancer. Cancer Gene Ther, 2008. 15(4): p. 231-40.
18. Meyerrose, T. E., et al., Lentiviral-transduced human mesenchymal stem cells persistently express therapeutic levels of enzyme in a xenotransplantation model of human disease. Stem Cells, 2008. 26(7): p. 1713-22.
19. McGinley, L., et al., Lentiviral vector mediated modification of mesenchymal stem cells & enhanced survival in an in vitro model of ischaemia. Stem Cell Res Ther, 2011. 2(2): p. 12.
20. Liang, X., et al., Human bone marrow mesenchymal stem cells expressing SDF-1 promote hematopoietic stem cell function of human mobilised peripheral blood CD34+ cells in vivo and in vitro. Int J Radiat Biol, 2010. 86(3): p. 230-7.
21. Glavaski-Joksimovic, A., et al., Glial cell line-derived neurotrophic factor-secreting genetically modified human bone marrow-derived mesenchymal stem cells promote recovery in a rat model of Parkinson's disease. J Neurosci Res, 2010. 88(12): p. 2669-81.
22. Liu, A. M., et al., Umbilical cord-derived mesenchymal stem cells with forced expression of hepatocyte growth factor enhance remyelination and functional recovery in a rat intracerebral hemorrhage model. Neurosurgery, 2010. 67(2): p. 357-65; discussion 365-6.
23. Yu, Y. S., et al., AKT-modified autologous intracoronary mesenchymal stem cells prevent remodeling and repair in swine infarcted myocardium. Chin Med J (Engl), 2010. 123(13): p. 1702-8.
24. Mueller, L. P., et al., TRAIL-transduced multipotent mesenchymal stromal cells (TRAIL-MSC) overcome TRAIL resistance in selected CRC cell lines in vitro and in vivo. Cancer Gene Ther, 2011. 18(4): p. 229-39.
25. Yan, C., et al., Suppression of orthotopically implanted hepatocarcinoma in mice by umbilical cord-derived mesenchymal stem cells with sTRAIL gene expression driven by AFP promoter. Biomaterials, 2014. 35(9): p. 3035-43.
26. Deng, Q., et al., TRAIL-secreting mesenchymal stem cells promote apoptosis in heat-shock-treated liver cancer cells and inhibit tumor growth in nude mice. Gene Ther, 2014. 21(3): p. 317-27.
27. Sage, E. K., et al., Systemic but not topical TRAIL-expressing mesenchymal stem cells reduce tumour growth in malignant mesothelioma. Thorax, 2014. 69(7): p. 638-47.
28. Lian, W. S., et al., In vivo therapy of myocardial infarction with mesenchymal stem cells modified with prostaglandin I synthase gene improves cardiac performance in mice. Life Sci, 2011. 88(9-10): p. 455-64.
29. Maijenburg, M. W., et al., Nuclear receptors Nur77 and Nurr1 modulate mesenchymal stromal cell migration. Stem Cells Dev, 2012. 21(2): p. 228-38.
30. Harper, M. M., et al., Transplantation of BDNF-secreting mesenchymal stem cells provides neuroprotection in chronically hypertensive rat eyes. Invest Ophthalmol Vis Sci, 2011. 52(7): p. 4506-15.
31. Zou, D., et al., In vitro study of enhanced osteogenesis induced by HIF-1alpha-transduced bone marrow stem cells. Cell Prolif, 2011. 44(3): p. 234-43.
32. Saito, S., et al., Mesenchymal stem cells stably transduced with a dominant-negative inhibitor of CCL2 greatly attenuate bleomycin-induced lung damage. Am J Pathol, 2011. 179(3): p. 1088-94.
33. Seo, K. W., et al., Anti-tumor effects of canine adipose tissue-derived mesenchymal stromal cell-based interferon-beta gene therapy and cisplatin in a mouse melanoma model. Cytotherapy, 2011. 13(8): p. 944-55.
34. Yang, H. M., et al., Enhancement of the immunosuppressive effect of human adipose tissue-derived mesenchymal stromal cells through HLA-G1 expression. Cytotherapy, 2012. 14(1): p. 70-9.
35. Liang, X. J., et al., Differentiation of human umbilical cord mesenchymal stem cells into hepatocyte-like cells by hTERT gene transfection in vitro. Cell Biol Int, 2012. 36(2): p. 215-21.
36. Fei, S., et al., The antitumor effect of mesenchymal stem cells transduced with a lentiviral vector expressing cytosine deaminase in a rat glioma model. J Cancer Res Clin Oncol, 2012. 138(2): p. 347-57.
37. Jaganathan, B. G. and D. Bonnet, Human mesenchymal stromal cells senesce with exogenous OCT4. Cytotherapy, 2012. 14(9): p. 1054-63.
38. Han, S. H., et al., Effect of ectopic OCT4 expression on canine adipose tissue-derived mesenchymal stem cell proliferation. Cell Biol Int, 2014. 38(10): p. 1163-73.
39. Shangguan, L., et al., Inhibition of TGF-beta/Smad signaling by BAMBI blocks differentiation of human mesenchymal stem cells to carcinoma-associated fibroblasts and abolishes their protumor effects. Stem Cells, 2012. 30(12): p. 2810-9.
40. Kearns-Jonker, M., et al., Genetically Engineered Mesenchymal Stem Cells Influence Gene Expression in Donor Cardiomyocytes and the Recipient Heart. J Stem Cell Res Ther, 2012. S1.
41. Ma, G. L., et al., [Study of inhibiting and killing effects of transgenic LIGHT human umbilical cord blood mesenchymal stem cells on stomach cancer]. Zhonghua Wei Chang Wai Ke Za Zhi, 2012. 15(11): p. 1178-81.
42. Huang, F., et al., Mesenchymal stem cells modified with miR-126 release angiogenic factors and activate Notch ligand Delta-like-4, enhancing ischemic angiogenesis and cell survival. Int J Mol Med, 2013. 31(2): p. 484-92.
43. Huang, F., et al., Overexpression of miR-126 promotes the differentiation of mesenchymal stem cells toward endothelial cells via activation of PI3K/Akt and MAPK/ERK pathways and release of paracrine factors. Biol Chem, 2013. 394(9): p. 1223-33.
44. Fang, Z., et al., Differentiation of GFP-Bcl-2-engineered mesenchymal stem cells towards a nucleus pulposus-like phenotype under hypoxia in vitro. Biochem Biophys Res Commun, 2013. 432(3): p. 444-50.
45. Madonna, R., et al., Transplantation of mesenchymal cells rejuvenated by the overexpression of telomerase and myocardin promotes revascularization and tissue repair in a murine model of hindlimb ischemia. Circ Res, 2013. 113(7): p. 902-14.
46. Zang, Y., et al., [Influence of CXCR4 overexpressed mesenchymal stem cells on hematopoietic recovery of irradiated mice]. Zhongguo Shi Yan Xue Ye Xue Za Zhi, 2013. 21(5): p. 1261-5.
47. Cao, Z., et al., Protective effects of mesenchymal stem cells with CXCR4 up-regulation in a rat renal transplantation model. PLoS One, 2013. 8(12): p. e82949.
48. Liu, S., et al., Overexpression of Wnt11 promotes chondrogenic differentiation of bone marrow-derived mesenchymal stem cells in synergism with TGF-beta. Mol Cell Biochem, 2014. 390(1-2): p. 123-31.
49. Zhu, Y., et al., Mesenchymal stem cell-based NK4 gene therapy in nude mice bearing gastric cancer xenografts. Drug Des Devel Ther, 2014. 8: p. 2449-62.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of selecting donor fibroblast cells and/or derivatives and/or vesicles thereof to provide to one or more recipient individuals, comprising the steps of:
identifying the expression of one or more human leukocyte antigens (HLA) on said donor fibroblast cells and/or derivatives and/or vesicles thereof; and
matching one or more recipient individuals to the donor fibroblast cells and/or derivatives and/or vesicles thereof based on the expression of one or more HLA in the recipient.

2. The method of claim 1, wherein the expression of the HLA is determined by nucleic acid levels, protein levels, or both.

3. The method of claim 1, wherein the HLA is HLA-A, HLA-B, HLA-C, HLA-C, HLA-DP, HLA-DQ, HLA-DR, HLA-B27, or a combination thereof.

4. The method of claim 1, wherein the expression of Twist is determined on the donor fibroblast cells and/or derivatives and/or vesicles thereof.

5. The method of claim 1, wherein the donor fibroblast cells and/or derivatives and/or vesicles thereof are further analyzed for one or more additional functional properties and/or one or more additional genotypes.

6. The method of claim 5, wherein the donor fibroblast cells and/or derivatives and/or vesicles thereof are analyzed for having one or more regenerative properties.

7. The method of claim 6, further defined as expressing one or more regenerative factors.

8. The method of claim 7, wherein the one or more regenerative factors is selected from the group consisting of interleukin (IL)-1, IL-3, granulocyte colony-stimulating factor (G-CSF), granulocyte macrophage colony-stimulating factor (GM-CSF), macrophage colony-stimulating factor (M-CSF), thrombopoietin (TPO), leukemia inhibitory factor, hepatic growth factor (HGF), brain derived neurotrophic factor (BDNF), nerve growth factor (NGF), connective tissue growth factor (CTGF), vascular endothelial growth factor (VEGF), fibroblast growth factor a (FGFa), fibroblast growth factor b (FGFb), platelet derived growth factor AA (PDGF-AA), platelet derived growth factor AB (PDGF-AB), angiopoietin, and a combination thereof.

9. The method of claim 6, wherein the donor fibroblast cells and/or derivatives and/or vesicles thereof are selected for a particular therapeutic application based on the expression of one or more regenerative or other factors.

10. The method of claim 9, wherein the therapeutic application is to stimulate hematopoiesis.

11. The method of claim 10, wherein the one or more regenerative or other factors are selected from the group consisting of interleukin-1 (IL-1), IL-3, granulocyte-colony stimulating factor (G-CSF), granulocyte-macrophage colony-stimulating factor (GM-CSF), thrombopoietin (TPO), leukemia inhibitory factor, and a combination thereof.

12. The method of claim 9, wherein the therapeutic application is to stimulate neurogenesis.

13. The method of claim 12, wherein the one or more regenerative or other factors are selected from the group consisting of brain derived neurotrophic factor (BDNF), nerve growth factor (NGF), connective tissue growth factor (CTCF), and a combination thereof.

14. The method of claim 9, wherein the therapeutic application is to stimulate angiogenesis.

15. The method of claim 14, wherein the one or more regenerative or other factors are selected from the group consisting of vascular endothelial growth factor (VEGF), fibroblast growth factor A (FGF-A), fibroblast growth factor B (FGF-B), platelet-derived growth factor AA (PDGF-AA), platelet-derived growth factor AB (platelet-derived growth factor AB), angiopoietin, and a combination thereof.

16. The method of claim 9, wherein the therapeutic application is to stimulate hepatic regeneration.

17. The method of claim 16, wherein the one or more regenerative or other factors is hepatic regeneration factor (HGF).

18. The method of claim 1, wherein the donor is a mammal.

19. The method of claim 18, wherein the mammal is a human, primate, murine, canine, feline, porcine, and/or bovine.

20. A composition of fibroblast cells and/or derivatives and/or vesicles thereof selected from the method of claim 1.

21. A pharmaceutical composition comprising the composition of claim 20 and a pharmaceutically acceptable carrier.

22. A method of treating a medical condition in an individual comprising the step of delivering a therapeutically effective amount of the pharmaceutical composition of claim 21 to the individual.

23. The method of claim 22, wherein the individual has or is at risk of having an inflammatory condition and/or a neurodegenerative condition and/or an autoimmune condition and/or a neoplastic condition and/or the frailty of aging.
